(19) United States
(12) Patent Application Publication
(10) Pub. No.: US 2012/0191075 A1
(43) Pub. Date: Jul. 26, 2012

(54) METHOD AND DEVICES FOR PULLING A TETHER THROUGH AN ORGAN WALL

(75) Inventor: Robert M. Trusty, Cincinnati, OH (US)

(73) Assignee: Ethicon Endo-Surgery, Inc., Cincinnati, OH (US)

(21) Appl. No.: 13/013,131

(22) Filed: Jan. 25, 2011

(51) Int. Cl. A61B 17/00

(52) U.S. Cl. 606/1

(57) ABSTRACT

A medical device is disclosed which can be utilized to access a surgical site within a patient. The medical device is equipped with an electronic tether having a connecting member at its free end. The connecting member has a free end and a body portion positioned between the free and the electronic tether. A tip at the free end of the connecting member has a smooth non-piercing leading end and surface contours suitable for engagement with a medical instrument. The body portion of the connecting member having a plurality of conductors, each conductor being separated from the adjacent conductor by one of a plurality of insulating members. At least one conductor transfers power from an external power source to the medical device and at least one conductor transferring information signals from the medical device to an external receiver.

### BACKGROUND

i. Field of the Invention

The present application relates to methods and devices for use in minimally invasive diagnostic, therapeutic, or surgical procedures and, more particularly, to devices for withdrawing a tether from a patient's body.

ii. Description of the Related Art

In minimally invasive diagnostic, therapeutic, and surgical procedures, such as laparoscopic surgery, a clinician may place one or more small ports into a patient's abdomen to gain access into the abdominal cavity of the patient. The clinician may use, for example, a port for insufflating the abdominal cavity to create space, a port for introducing a laparoscope for viewing, and a number of other ports for introducing diagnostic, therapeutic, or surgical instruments for viewing, sampling, treating, or operating on tissue. Other minimally invasive surgical procedures include natural orifice transluminal endoscopic surgery (NOTES™) wherein surgical instruments and viewing devices are introduced into a patient's body through, for example, the mouth, nose, vagina, or rectum. The benefits of minimally invasive procedures compared to open surgery procedures for treating certain types of wounds and diseases are now well-known to include faster recovery time and less pain for the patient, better outcomes, and lower overall costs.

In minimally invasive surgery, it is desirable to deliver devices that can be used for surgery into the patient's body via one of the access ports without occupying the port so that other devices may use the space. Some of the devices delivered through the port may be electronic in nature and require power or electronic data to be delivered to them to operate, for example to power a light source or to adjust the focus of a lens system. They may also need to deliver electronic information to personnel in the operating room, for example an image stream. Other devices may make use of an electronic connector, such as a motorized stapler, RF ablation device, harmonic scalpel, bi-polar forceps, and the like.

The foregoing discussion is intended only to illustrate various aspects of the related art in the field of the invention at the time, and should not be taken as a disavowal of claim scope.

### SUMMARY

One way of delivering power or data signals to devices that have been inserted into a patient and for delivering information from such an internal device back to personnel in the operating room is for the delivered device to include an electronic tether having a connecting member on a free end. Once delivered into the patient, a way of bringing the electronic tether and connecting member out of the body must be provided, unless the tether were to occupy port space or exit adjacent to a port. However, for pure NOTES™ procedures, no port may exist. In this case, it would be useful to be able to insert a small device, such as a 2 mm trocar and grasper, suture, a rigid hook, a threaded rod, or the like, through the abdominal wall to pull the tether outside the body.

A device for use in medical procedures with other medical devices and instruments is described herein. In certain embodiments, the device includes a connecting member having a free end, a joined end, and a body portion positioned between the free and joined ends. The joined end is attached to an electronic tether of a first medical device or instrument. The free end has a tip which has a smooth non-piercing leading end and surface contours suitable for engagement with a second medical instrument. The body portion has at least one conducting portion, and may have a plurality of conducting portions, wherein each of the plurality of conducting portions is separated from an adjacent conducting portion by one of a plurality of insulating portions.

In various embodiments, the smooth leading end of the tip may be in the form of a truncated cone having a flattened end. The body portion may define a longitudinal axis.

In at least one embodiment, the surface contours of the tip form a shoulder portion adjacent the body portion of the connecting member, a neck portion, and a head portion. The shoulder portion is beveled inwardly toward the neck portion, which is sized, in cross section, to be smaller than the size in cross-section of the body and head portions.

In another embodiment, the surface contours form a notch cut into the tip. In a further embodiment, the surface contours of the tip may form a bore passing in a direction transverse to the longitudinal axis. In another embodiment, the surface contours of the tip form a bore cut into the truncated cone in a direction coaxial to the longitudinal axis.

In certain embodiments, a medical device having an electronic tether attached thereto may be provided, wherein the tether includes a connecting member, as described in any of the foregoing embodiments, attached to the free end of the tether.

Certain embodiments of the connector may be characterized as a small diameter, multi-conductor connector that has a low profile and a smooth exterior surface, and that includes surface contours in the tip of the connector to aid a grasper in securely engaging the connector to pull it through the abdominal wall.

### FIGURES

Various features of the embodiments described herein are set forth with particularity in the appended claims. The various embodiments, however, both as to organization and methods of operation, together with advantages thereof, may be understood in accordance with the following description taken in conjunction with the accompanying drawings as follows.

FIGS. 1A-D show alternative embodiments of connector tip shapes for aiding a grasper in holding and pulling a connecting member.

FIG. 2 shows the embodiment of a tip shape shown in FIG. 1 A connected to a joining member and the wire.

FIG. 3 shows a view of the external surface of a patient's abdomen during a laparoscopic procedure with a trocar inserted through an opening in an access port.

FIG. 4 shows a laparoscopic view of the inside of a patient's abdomen with the embodiment of the connector tip of FIG. 2 being pulled through the abdominal wall with a pair of graspers.

FIG. 5 shows the external view of the view of FIG. 4 with the connecting member of FIG. 2 being pulled through the abdominal wall of a patient.

Corresponding reference characters indicate corresponding parts throughout the several views. The exemplifications set out herein illustrate various embodiments of the invention, in one form, and such exemplifications are not to be construed as limiting the scope of the invention in any manner.

### DESCRIPTION

Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located farthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

“Small diameter” as used herein means about 2.5 mm or less. “Low profile” as used herein means that the features and contours on the surface of the devices are smooth and radiused to avoid becoming caught in tissue and to avoid causing any drag or resistance when being pulled through a wall of tissue.

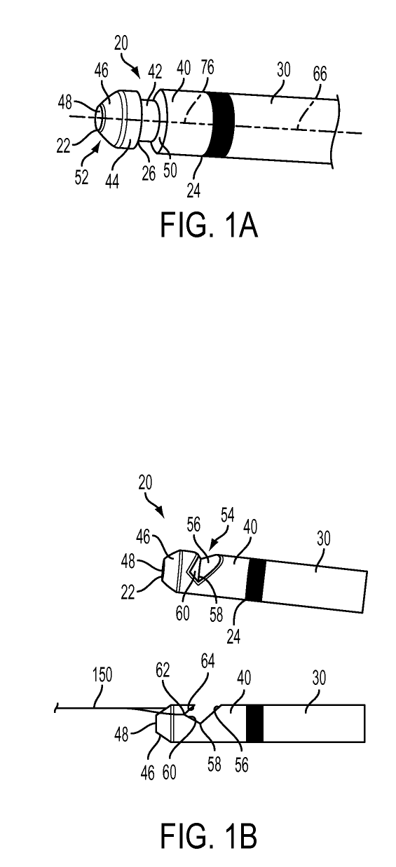

FIG. 1 is a view of four exemplary tip 20 options on the end of the connector barrel 30 of a connecting member having various embodiments of engagement surfaces to allow a grasper, suture, or the like to securely engage the connector to pull it to the desired location.

One embodiment of a tip 20 is shown in FIG. 1A, wherein the tip 20 includes a body portion 40 that is joined at its trailing end 24 to a connector barrel 30. Tip 20 also includes in this embodiment, a shoulder portion 50 following the body 40. Shoulder portion 50 is beveled inwardly toward a neck portion 42 that is smaller in diameter than the body 40. Adjacent the neck portion 42 is a head portion 52. The head portion includes an annular portion 44, leading to a truncated cone portion 46 having a flattened end portion 48 at the leading end 22 of tip 20. Annular portion 44 is larger in diameter than neck portion 42 and may be generally the same diameter as the body portion 40. There is an annular rim 26 that extends outwardly from neck portion 42 at a sharp angle, preferably at an angle of ninety degrees or less to form an acute angle to aid in grasping the tip 20. Connector barrel 30 and body 40 may be similarly sized with the same or generally the same diameters, or may have differently sized diameters provided that the transition from connector barrel 30 to body 40 is smooth to form a low profile. Barrel 30 may be tubular in shape, having a longitudinal central axis 66. Body 40 is preferably also tubular in shape and has a longitudinal central axis 76 that aligns with the axis 66 of barrel 30.

FIG. 1B illustrates an alternative embodiment of a tip 20 connected, in coaxial alignment, to barrel 30. The truncated cone portion 46 and the flattened end 48 at the leading end of tip 20 are the same or similar to the like portions in FIG. 1A, however, there is no neck or shoulder portions. The trailing end 24 of body 40 extends from the end of barrel 30 to the cone portion 46. The engagement surface in this embodiment is a notch 54 cut into body portion 40. Notch 54 includes a first slope 56 running downwardly from the surface of body portion 40 to a valley 58. The valley is followed by a second slope 60 running upwardly from the valley 58 to a groove 62 which is followed by a third slope 64 running upwardly to the surface of body portion 40. The first slope and the third slope are generally parallel to each other and may be parallel to each other. The second slope lies on a plane transverse to the plane of the first and second slopes. The valley 58, second and third slopes 60, 64, and the groove 62 form an engagement surface that enables a grasper, a suture, or the like to securely hold the tip 20 to pull the connector in a desired direction. The engagement surface, when pulled through tissue in the direction of the leading end 22 of the connector, will not catch tissue because the angles of the valley 58, second and third slopes 60, 64, and the groove 62 are away from the tissue. Tissue may contact first slope 56 as the connector is pulled through tissue, but slope 56 provides a smooth surface and smoothly transitions to the surface of body 40.

FIG. 1C illustrates an embodiment of tip 20 wherein body portion 40 extends coaxially from its trailing end 24 on the end of barrel 30 to the truncated cone portion 46 and flattened end 48 on the leading end 22 of tip 20. The engagement surface in this embodiment is a bore 70 passing through body portion 40 in a direction transverse to central longitudinal axis 76.

Another embodiment of tip 20 is shown in FIG. 1D, wherein body portion 40 extends coaxially from its trailing end 24 on the end of barrel 30 to the truncated cone portion 46. There are no necks, notches, or bores cut into body portion 40. The engagement surface in this embodiment is an axial bore 72 cut into the leading end 22 of tip 20 in the place of flattened end 48 of the other embodiments. Bore 72 has threads 74 into which a male extension having complementary threads on the end of a device may be attached to pull the connector in a desired direction.

It will be understood by those normally skilled in the art that numerous other geometries of tips 20 and grasping devices that aid in attachment to devices that are capable of pulling the connector tip 20 through an organ or body wall may be developed. For example, a collet-like grasping device which uses diametrical pressure to impress shaped fingers around the exterior surface of a connector tip formed in the negative of the grasping tip interior shape.

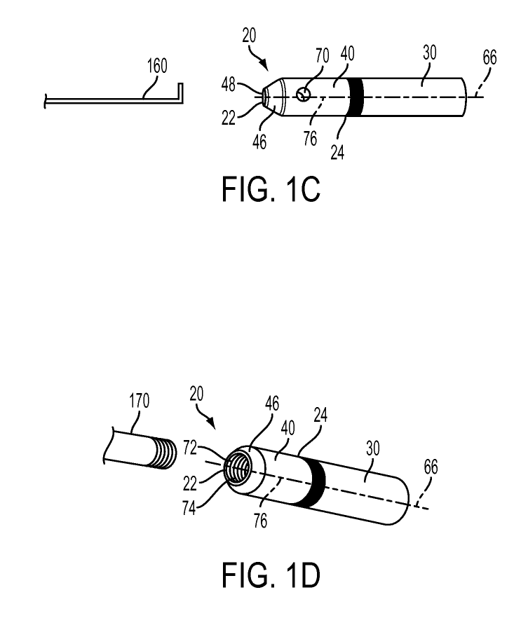

FIG. 2 shows the tip 20 and connector barrel 30 connected to a joining member 80, which in turn is attached to an electronic tether, in the form of a wire 32. Connector barrel 30 is shown having a plurality of insulated rings 82, each separating one of a plurality of conductors. The insulated rings 82 are made of a non-conducting material and the conductors are made of a conducting material, such as stainless steel, titanium, or another suitable conducting material. For example, there may be four metal conductors, two for outwardly directed video signals and two for inwardly directed power signals. An exemplary power signal may be used to power a medical device on the other end of the electronic tether, such as a camera, while another power signal may be used to power a light source, such as a light emitting diode (LED) for the camera. The camera may send +/− video or imaging signals outwardly from the connector. A knurled ring 34 and flange 78 at the trailing end of connector barrel 30 provide a tactile surface to aid in manually connecting joining member 80 to connector barrel 30.

The joining member 80 may be releasably attached to the electronic tether, or may form an integral part thereof. The connecting barrel 30 may be releasable attached to the joining member 80 or may form an integral part thereof. The releasable attachment may be by any suitable known means that will securely attached the various components during use, but permit detachment upon removal for disposal or sterilization prior to re-use, as appropriate.

A method is provided for using the connecting member described herein in minimally invasive procedures to electrically connect an internal medical device to an external device. The external device may operatively connect the connecting member, and thereby the internal medical device to a power source, by a direct connection to an electric outlet or indirectly through another device, or may provide power by means of a battery. The external device may have one or both of electrical connectors for delivering power or data signals to the internal medical device and receivers for receiving signals from the internal device. The method may include in general, the step of equipping the internal medical device with an electronic tether having a free end, wherein the free end of the electronic tether has a connecting member. The connecting member may have, as described herein, a free end and a body portion positioned between the free end and the electronic tether. The free end of the connecting member has a tip which has a smooth non-piercing leading end and surface contours suitable for engagement with a medical instrument. The body portion of the connecting member has at least one conducting portion. Equipping the medical device may include any one or more of the following steps: attaching a non-releasable tether having a tip, attaching a releasably attachable electronic tether to the medical device, attaching a releasably attachable connecting member to the electronic tether, or attaching a releasably attachable tip to the body portion of the connecting member.

The method may, in general, further include the steps of inserting the internal medical device into a body cavity of a patient through a first opening, creating a second opening and a path into the body cavity, inserting a first medical instrument which has an engagement portion through the path into the body cavity, engaging the engagement surfaces of the tip of the connecting member with the engagement portion of the first medical instrument, withdrawing the connecting member and a portion of the electronic tether from the body cavity of the patient through the path with the first medical instrument, and, connecting the connecting member to a device external to the patient. The step of creating a second opening and a path into the body cavity preferably includes making an incision with, and inserting into the body cavity, a combination of a trocar and an obturator. After inserting the combination of the trocar and the obturator through the incision, the obturator is withdrawn prior to inserting the first medical instrument into the body cavity.

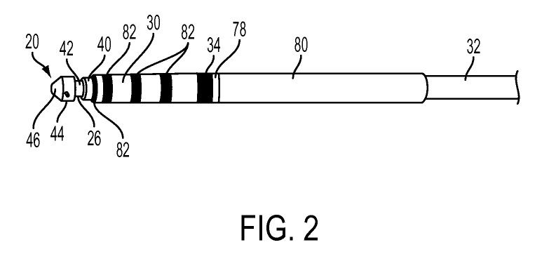

In use, with specific reference to the embodiments described herein, a tethered medical device, such as a camera, may be inserted into an internal cavity in a patient 10, such as the abdominal cavity 14, through a small, key-hole incision, with a laparoscope, or through a natural orifice. An open ended tube 92 may be inserted into the abdominal cavity. The tethered medical device is placed into the abdominal cavity 14. FIG. 3 shows clinicians inserting a tubular medical instrument 92 through the exterior surface 12 of a patient 10 through one of the access ports in an access device 90 to enter the abdominal cavity 14 of a patient 10 during a procedure. A secondary opening 16 and path into the abdominal cavity 14 are made using, for example, a small needle or a small trocar 200 (e.g., about 2.5 mm in diameter) and obturator. The obturator is then removed, leaving the trocar 200 in place. Through the trocar 200, another medical instrument, such as a grasper, suture, or the like, having a diameter smaller than that of the trocar 200 (e.g., less than 2.5 mm in diameter) is inserted into the abdominal cavity 14.

Those skilled in the art will appreciate that the tethered medical device may be a device other than a camera or other imaging devices. The medical device may be a motorized stapler, RF ablation device, harmonic scalpel, bi-polar forceps, electrocautery device, and the like. The electronic tether may be releasably attached to the medical device, or may be integrally attached in any suitable known manner.

A medical instrument selected from the group consisting of a grasper, a suture, a rigid hook, and a threaded rod may be used to withdraw the connector and a portion of the tether from the patient's body cavity.

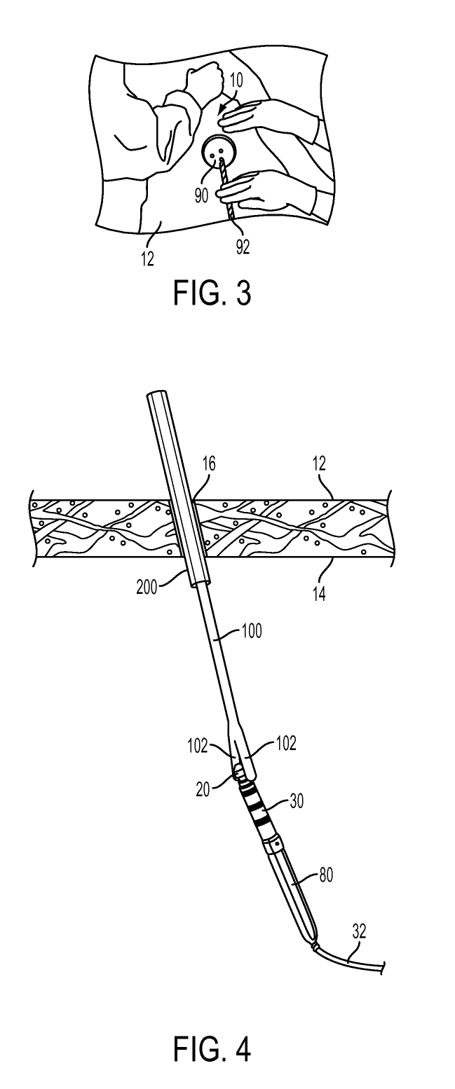

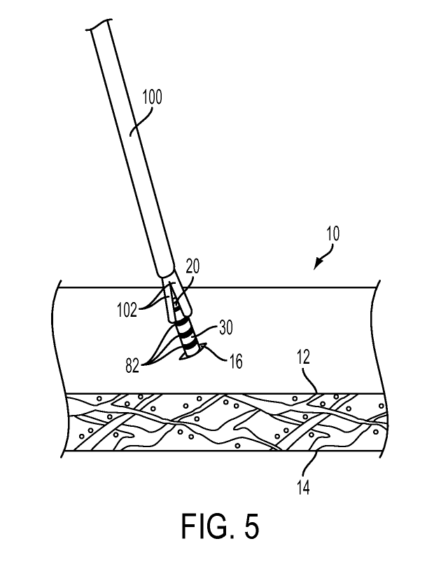

Referring to FIGS. 4 and 5, a grasper 100 having movable jaws 102 is passed through the trocar 200 in the transcutaneous incision 16 in the patient's abdomen. The jaws 102 of the grasper 100 are opened to place tip 20 between the jaws 102. The jaws 102 grasp the engagement surface of tip 20, such as the tip 20 shown in FIG. 1A, to securely hold tip 20 to pull the connecting member, and with it a portion of the electronic tether 32, back until contacting the distal tip of the trocar, then pulling the trocar, grasper, connecting member, and portion of the electronic tether through the incision 16 out of the abdominal cavity 14 to the external surface 12 of the patient 10, as shown in FIG. 5. Alternatively, the grasper jaws 102 could be replaced with a suture loop 150 to snare the tip shown in FIG. 1B or a rigid hook 160 to be inserted in the transverse hole shown in FIG. 1C or a male-threaded rod 170 to be screwed into the female threads shown in FIG. 1D. Once the connecting member is outside of the patient, it can be connected to the external device and through the device, to a power source and at least one receiver for sending power to, and receiving the signals from, the conductors. The receiver may be any suitable known receiver for receiving signals and, in various embodiments, may be operatively connected to any suitable known controller for converting the received signals, for example, into observable images or data.

The embodiments of the devices described herein may be introduced inside a patient using minimally invasive or open surgical techniques. In some instances it may be advantageous to introduce the devices inside the patient using a combination of minimally invasive and open surgical techniques. Minimally invasive techniques may provide more accurate and effective access to the treatment region for diagnostic and treatment procedures. To reach internal treatment regions within the patient, the devices described herein may be inserted through natural openings of the body such as the mouth, nose, anus, and/or vagina, for example. Minimally invasive procedures performed by the introduction of various medical devices into the patient through a natural opening of the patient are known in the art as NOTES™ procedures. Some portions of the devices may be introduced to the tissue treatment region percutaneously or through small-keyhole-incisions.

Endoscopic minimally invasive surgical and diagnostic medical procedures are used to evaluate and treat internal organs by inserting a small tube into the body. The endoscope may have a rigid or a flexible tube. A flexible endoscope may be introduced either through a natural body opening (e.g., mouth, anus, and/or vagina) or via a trocar through a relatively small-keyhole-incision incisions (usually 0.5-2.5 cm). The endoscope can be used to observe surface conditions of internal organs, including abnormal or diseased tissue such as lesions and other surface conditions and capture images for visual inspection and photography. The endoscope may be adapted and configured with working channels for introducing medical instruments to the treatment region for taking biopsies, retrieving foreign objects, and/or performing surgical procedures.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK®bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. Other sterilization techniques can be done by any number of ways known to those skilled in the art including beta or gamma radiation, ethylene oxide, and/or steam.

Although the various embodiments of the devices have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A device for use in medical procedures with a medical device and medical instruments comprising:
a connecting member having a free end, a joined end, and a body portion positioned between the free and joined ends;
wherein the joined end is attached to an electronic tether of a medical device and the free end has a tip having a smooth non-piercing leading end and surface contours suitable for engagement with a medical instrument, and
wherein the body portion has at least one conducting portion.

2. The device of claim 1 wherein the body portion has a plurality of conducting portions, each conducting portion separated from an adjacent conducting portion by one of a plurality of insulating portions.

3. The device of claim 1 wherein the body portion defines a longitudinal axis and the surface contours of the tip form a shoulder portion adjacent the body portion, a neck portion, and a head portion, the shoulder portion being beveled inwardly toward the neck portion being sized in cross section smaller than the size in cross-section of the body and head portions.

4. The device of claim 3 wherein the head portion includes an annular ring leading to a truncated cone having a flattened end at the leading end of the tip, and the neck portion has an annular rim that extends outwardly from neck portion to the annular ring of the head portion at an angle sufficient to aid engagement with the second instrument.

5. The device of claim 1 wherein the surface contours form a notch cut into the tip.

6. The device of claim 5 wherein the tip has a body and the notch includes a first slope running downwardly from the surface of tip body portion to a valley, a second slope running upwardly from the valley, a groove adjacent the second slope, and a third slope running upwardly from the groove to the surface of tip body portion.

7. The device of claim 6 wherein the first slope and the third slope are parallel to each other and the second slope lies on a plane transverse to the plane of the first and second slopes.

8. The device of claim 7 wherein the smooth leading end of the tip comprises a truncated cone having a flattened end.

9. The device of claim 1 wherein the connecting member defines a longitudinal axis and the surface contours of the tip form a bore passing in a direction transverse to the longitudinal axis.

10. The device of claim 9 wherein the smooth leading end of the tip comprises a truncated cone having a flattened end.

11. The device of claim 1 wherein the connecting member defines a longitudinal axis and the surface contours of the tip form a truncated cone and a bore cut into the truncated cone in a direction coaxial to the longitudinal axis.

12. The device of claim 11 wherein the bore is threaded for threaded engagement with the second instrument.

13. The device of claim 1 wherein the medical instrument is selected from the group consisting of a grasper, a suture, a rigid hook, and a threaded rod.

14. A method for use in minimally invasive procedures to electrically connect an internal medical device to an external device comprising:
equipping a first medical device with an electronic tether having a free end, the free end of the electronic tether having a connecting member, the connecting member having a free end and a body portion positioned between the free end and the electronic tether, the free end of the connecting member having a tip having a smooth non-piercing leading end and surface contours suitable for engagement with a medical instrument, and, the body portion of the connecting member having at least one conducting portion;
inserting the first medical device into a body cavity of a patient through a first opening;
creating a second opening and a path into the body cavity;
inserting a first medical instrument through the path into the body cavity, the first medical instrument having an engagement portion;
engaging the engagement surfaces of the tip of the connecting member with the engagement portion of the first medical instrument;
withdrawing the connecting member and a portion of the electronic tether with the first medical instrument from the body cavity of the patient through the path; and,
connecting the connecting member to a device external to the patient.

15. The method of claim 14 wherein the medical instrument is selected from the group consisting of a grasper, a suture, a rigid hook, and a threaded rod.

16. The method of claim 14 wherein the first medical device is a camera.

17. The method of claim 14 wherein the connecting member includes a plurality of conductors, each conductor being separated from the adjacent conductor by one of a plurality of insulating members and wherein there is at least one conductor for transferring power to the first medical device and at least one conductor for transferring information signals from the first medical device to an external receiver.

18. The method of claim 14 wherein the step of creating a second opening and a path into the body cavity further comprises making an incision with and inserting into the body cavity a combination of a trocar and an obturator.

19. The method of claim 18 further comprising following the step of inserting the combination of the trocar and the obturator through the incision, withdrawing the obturator prior to inserting the first medical instrument into the body cavity.

20. A medical device comprising:
an electronic tether having a free end, the free end of the electronic tether having a connecting member, the connecting member having a free end and a body portion positioned between the free and the electronic tether, the free end of the connecting member having a tip having a smooth non-piercing leading end and surface contours suitable for engagement with a medical instrument, and,
wherein the body portion of the connecting member has a plurality of conductors, each conductor being separated from the adjacent conductor by one of a plurality of insulating members, at least one conductor structured for transferring power from an external power source to the medical device and at least one conductor structured for transferring information signals from the medical device to an external receiver.
